(19) United States
(12) Patent Application Publication
(10) Pub. No.: US 2016/0089032 A1
(43) Pub. Date: Mar. 31, 2016

(54) PROBE ASSEMBLY AND END COVER

(71) Applicant: OLYMPUS CORPORATION, Tokyo (JP)

(72) Inventors: Seiki TORIYAMA, Hino-shi (JP); Yoshimine KOBAYASHI, Hachioji-shi (JP)

(21) Appl. No.: 14/864,343

(22) Filed: Sep. 24, 2015

Related U.S. Application Data
(60) Provisional application No. 62/057,665, filed on Sep. 30, 2014.

(51) Int. Cl. A61B 5/00; A61B 1/07; A61B 1/06; A61B 1/00

(52) U.S. Cl. CPC A61B 5/0084; A61B 1/00096; A61B 1/07; A61B 1/00045; A61B 1/0684; A61B 1/00009; A61B 1/0646

(57) ABSTRACT

A probe assembly includes: a measurement probe having a plurality of optical fibers configured to propagate light, emit the light to a measurement target, and receive scattered light returned from the measurement target; and an end cover having: an endoscope holding portion configured to hold an insertion portion of an endoscope, the insertion portion being configured to be inserted into a body cavity of a subject; and a probe position defining portion configured to define a position of a distal end surface of the plurality of optical fibers of the measurement probe.

### CROSS-REFERENCE TO RELATED APPLICATIONS

This application is based upon and claims the benefit of priority from U.S. Provisional Patent Application No. 62/057,665, filed on Sep. 30, 2014, the entire contents of which are incorporated herein by reference.

### BACKGROUND

1. Technical Field

The disclosure relates to a probe assembly including a measurement probe that measures optical characteristics of a body tissue and an end cover that defines a position of the measurement probe, and relates to an end cover that defines a distal end position of the measurement probe.

2. Related Art

In recent years, an optical measurement device is known which emits illumination light to a body tissue and estimates characteristics of the body tissue based on measured values of detected light reflected or scattered from the body tissue. The optical measurement device is used by being combined with an endoscope that observes an organ such as a digestive organ. As such an optical measurement device, an optical measurement device using Low-Coherence Enhanced Backscattering (LEBS) is proposed, which detects characteristics of a body tissue by emitting low-coherent white light with a short spatial coherence length to the body tissue from an illumination fiber of a measurement probe, detecting scattered light entering at angles different from each other by using a plurality of light-receiving fibers, and measuring intensity distribution of the scattered light by using spectroscopes, each of which is provided for each of the light-receiving fibers (for example, see Japanese Patent No. 5049415).

### SUMMARY

In some embodiments, a probe assembly includes: a measurement probe having a plurality of optical fibers configured to propagate light, emit the light to a measurement target, and receive scattered light returned from the measurement target; and an and cover having: an endoscope holding portion configured to hold an insertion portion of an endoscope, the insertion portion being configured to be inserted into a body cavity of a subject; and a probe position defining portion configured to define a position of a distal end surface of the plurality of optical fibers of the measurement probe.

In some embodiments, an end cover includes: an endoscope holding portion configured to hold an insertion portion of an endoscope, the insertion portion being configured to be inserted into a body cavity of a subject; and a probe position defining portion configured to define a position of a distal end surface of a plurality of optical fibers of a measurement probe, the plurality of optical fibers being configured to propagate light, emit the light to a measurement target, and receive scattered light returned from the measurement target.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic diagram illustrating a schematic configuration of an endoscope system according to a first embodiment of the present invention;

FIG. 2 is a block diagram schematically illustrating a functional configuration of an optical measurement system according to the first embodiment of the present invention;

FIG. 3 is a diagram schematically illustrating a configuration of an insertion portion and an end cover of an endoscope according to the first embodiment of the present invention;

FIG. 4 is a plan view schematically illustrating a configuration of the end cover according to the first embodiment of the present invention;

FIG. 5 is a diagram schematically illustrating a configuration of a distal end portion and the end cover of the endoscope according to the first embodiment of the present invention when a measurement probe is inserted;

FIG. 6 is a diagram schematically illustrating a configuration of an insertion portion and an end cover of an endoscope according to a second embodiment of the present invention;

FIG. 7 is a diagram schematically illustrating a configuration of a distal end portion and the end cover of the endoscope according to the second embodiment of the present invention when a measurement probe is inserted;

FIG. 8 is a diagram schematically illustrating a configuration of an insertion portion of an endoscope, a distal end portion of a measurement probe, and an end cover according to a first modified example of the second embodiment of the present invention;

FIG. 9 is a diagram schematically illustrating a configuration of an insertion portion of an endoscope, a distal end portion of a measurement probe, and an end cover according to a second modified example of the second embodiment of the present invention;

FIG. 10 is a schematic diagram illustrating a schematic configuration of an endoscope system according to a third embodiment of the present invention;

FIG. 11 is a perspective view schematically illustrating a configuration of an insertion portion of an endoscope, a distal end portion of a measurement probe, and an end cover according to the third embodiment of the present invention; and

FIG. 12 is a partial cross-sectional view taken along line A-A in FIG. 11.

### DETAILED DESCRIPTION

Modes for carrying out the invention (hereinafter referred to as “embodiments”) will be described below with reference to the drawings. The same reference signs are used to designate the same elements throughout the drawings. The drawings are schematic, and the relation between the thickness and the width of each member, and the ratio of the size of each member should be noted that this is different from the reality. Also in between the drawings, portions in which dimensional relationships and ratios are different from each other are included. The present invention is not limited by the embodiments.

### First Embodiment

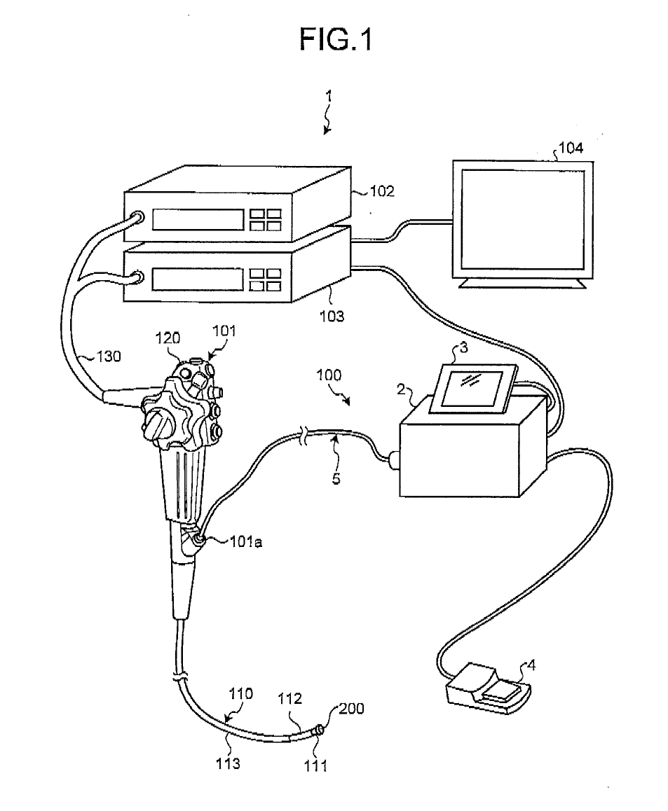

FIG. 1 is a schematic diagram illustrating a schematic configuration of an endoscope system according to a first embodiment of the present invention. The endoscope system 1 includes an optical measurement system 100 that performs an optical measurement on a measurement target such as a body tissue that is a scatterer and detects characteristics (characteristic features) of the measurement target, an endoscope 101 that captures an in-vivo image of a subject by inserting its distal end portion into a body cavity of the subject, a light source device 102 that generates illumination light emitted from the distal end portion of the endoscope 101, a processing device 103 that performs predetermined image processing on the in-vivo image captured by the endoscope 101 and generally controls an operation of the entire endoscope system 1, and a display device 104 that displays the in-vivo image on which the processing device 103 performs image processing.

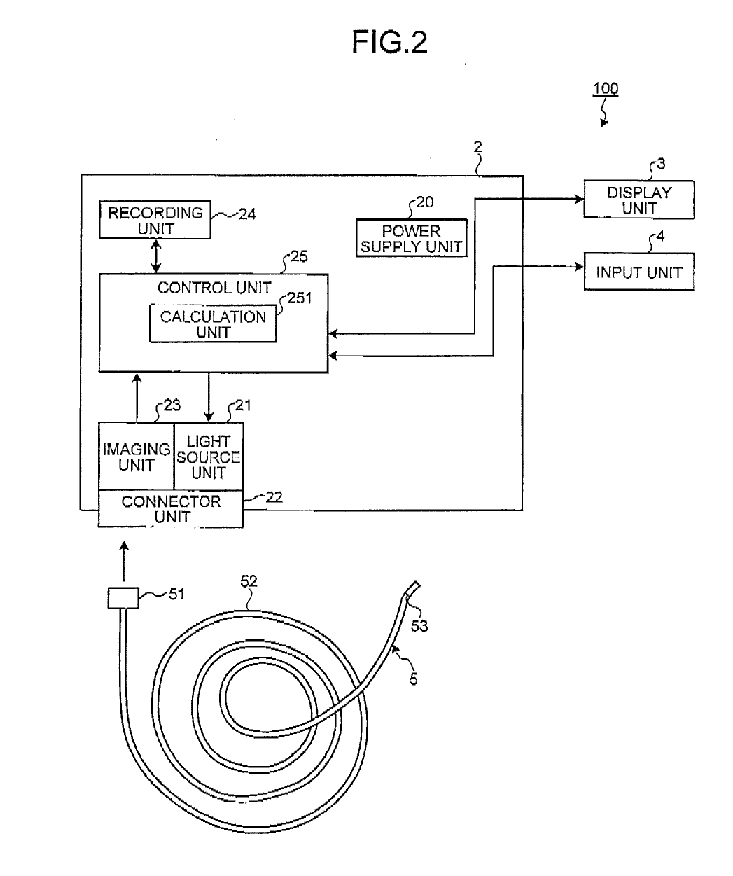

FIG. 2 is a block diagram schematically illustrating a functional configuration of the optical measurement system according to the embodiment of the present invention. The optical measurement system 100 illustrated in FIGS. 1 and 2 includes an optical measurement device 2, a display unit 3 that displays a measurement result of the optical measurement device 2, an input unit 4 that receives an input of an instruction signal that instructs the optical measurement device 2 to perform measurement, and a measurement probe 5 which is attachable to and detachable from the optical measurement device 2 and which is inserted into the subject.

First, a configuration of the optical measurement device 2 will be described. The optical measurement device 2 includes a power supply unit 20, a light source unit 21, a connector unit 22, an imaging unit 23, a recording unit 24, and a control unit 25. The power supply unit 20 supplies power to each unit of the optical measurement device 2.

The light source unit 21 emits illumination light to the measurement probe 5 through the connector unit 22. The light source unit 21 is realized by using, for example, a light-emitting element that is an incoherent light source such as a white LED (Light Emitting Diode), a condenser lens that condenses light emitted by the light-emitting element, and a filter that transmits light of a predetermined wavelength band. Examples of the condenser lens include a condenser lens and a collimator lens. The light source unit 21 emits incoherent light having at least one spectral component to the measurement probe 5 through the connector unit 22 as illumination light. The light-emitting element may be realized by using an incoherent light source such as a xenon lamp, a tungsten lamp, and a halogen lamp.

The connector unit 22 attachably and detachably connects the measurement probe 5 to the optical measurement device 2.

The imaging unit 23 generates an electrical signal by receiving return light of the illumination light which is emitted from a distal end of the measurement probe 5 and reflected and/or scattered from a measurement target and performing photoelectric conversion and outputs the electrical signal to the control unit 25. The imaging unit 23 is realized by using an imaging element such as a Charge Coupled Device (CCD) image sensor and a Complementary Metal Oxide Semiconductor (CMOS) image sensor. The imaging unit 23 can also be realized by a simple photoelectric conversion element such as a photodiode in addition to the imaging element.

The recording unit 24 records various programs to operate the optical measurement device 2 and various data and various parameters used by the optical measurement device 2. The recording unit 24 is realized by using a volatile memory, a non-volatile memory, and the like. The recording unit 24 temporarily records information and data that are being processed by the optical measurement device 2. Further, the recording unit 24 records a measurement result of the optical measurement device 2 and information related to an arrangement of a light receiving fiber. The recording unit 24 may be configured by using a memory card or the like attached from the outside of the optical measurement device 2.

The control unit 25 controls processing operation of each unit of the optical measurement device 2. The control unit 25 is configured by using a Central Processing Unit (CPU) and the like and generally controls the optical measurement device 2 by transmitting instruction information and data to each component of the optical measurement device 2. The control unit 25 includes a calculation unit 251. The calculation unit 251 performs a plurality of calculation processes based on the electrical signal inputted from the imaging unit 23 and calculates characteristic values related to the characteristics of the measurement target.

The display unit 3 outputs various information of the optical measurement device 2. Specifically, the display unit 3 outputs information inputted from of the optical measurement device 2. The display unit 3 is realized by using a display panel such as a liquid crystal display panel and an organic Electro Luminescence (EL) display panel, a speaker, and the like. A touch panel that receives an input of a position signal corresponding to a position contacted from the outside may be provided on a display screen of the display unit 3.

The input unit 4 receives an input of an instruction signal that instructs the optical measurement device 2 to perform measurement. The input unit 4 is realized by using, for example, a foot switch. In addition, the input unit 4 may be realized by an input interface such as a keyboard and a mouse or may be realized by combining these components.

The measurement probe 5 is configured by using at least a plurality of optical fibers. Specifically, the measurement probe 5 is realized by using an illumination fiber (an illumination channel) that emits illumination light to a measurement target and a plurality of light receiving fibers (light receiving channels) which the return light (scattered light) of the illumination light reflected and/or scattered from the measurement target enters at different angles. The measurement probe 5 includes a proximal end portion 51 that is attachably and detachably connected to the connector unit 22 of the optical measurement device 2, a flexible portion 52 having flexibility, and a distal end portion 53 in which a distal end surface of a fiber group including the illumination fiber and the light receiving fibers is exposed from its distal end surface and which emits the illumination light supplied from the light source unit 21 through the connector unit 22 and receives the return light of the illumination light from the measurement target.

In the optical measurement system 100 configured as described above, the measurement probe 5 is inserted through a treatment tool channel 101a of the endoscope 101 (scope of endoscope) of the endoscope system 1, the illumination fiber emits the illumination light to the measurement target, and each of the plurality of light receiving fibers receives the return light of the illumination light reflected and/or scattered from the measurement target at the distal end portion 53 and emits the return light to each light receiving surface of the imaging unit 23. Thereafter, the calculation unit 251 calculates the characteristic values of the characteristics of the measurement target based on a measurement result of each light receiving surface of the imaging unit 23.

The endoscope 101 includes an insertion portion 110 having an elongated shape with flexibility, an operating unit 120 which is connected to the proximal end of the insertion portion 110 and receives inputs of various operation signals, and a universal cord 130 which extends in a direction different from a direction in which the insertion portion 110 extends from the operating unit 120 and includes various cables connected to the light source device 102 and the processing device 103.

The insertion portion 110 includes a distal end portion 111 including an imaging element in which pixels that generate signals by receiving light and performing photoelectric conversion are two-dimensionally arranged, a bendable bending portion 112 configured by a plurality of bending pieces, and a flexible tube portion 113 which has flexibility and is connected to the proximal end of the bending portion 112.

In the first embodiment, an end cover 200 is attached to the distal end portion 111 of the insertion portion 110. The end cover 200 is fitted to the distal end portion 111, so that the end cover 200 is attached to the distal end portion 111.

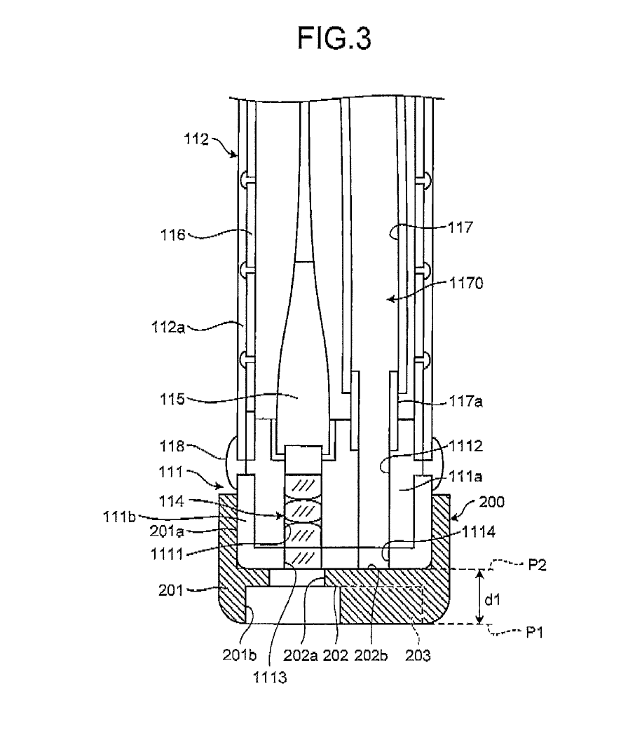

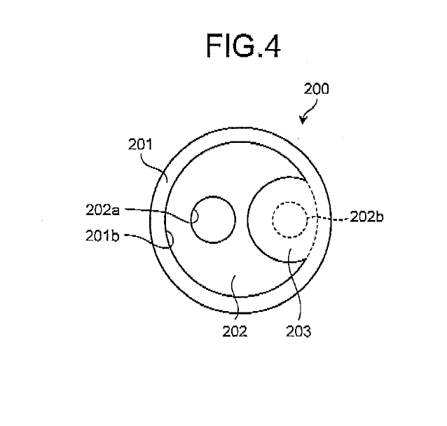

Here, a configuration of the insertion portion 110 and the end cover 200 of the endoscope 101 will be described with reference to the drawings. FIG. 3 is a diagram schematically illustrating a configuration of the distal end portion and the end cover of the endoscope according to the first embodiment. FIG. 4 is a plan view schematically illustrating a configuration of the end cover according to the first embodiment.

The distal end portion 111 includes a distal end main body 111a and a main body cover 111b. Two through holes (through holes 1111 and 1112) are formed in the distal end main body 111a. In the through hole 1111, a lens group 114 including a plurality of lenses and one end (an end of a light receiving surface) of an imaging element 115 are held. The imaging element 115 is realized by using an imaging element such as a CCD image sensor and a CMOS image sensor.

The main body cover 111b has a bottomed tubular shape and is attached by being fitted to the outer circumference of the distal end main body 111a. Two through holes (through holes 1113 and 1114) are formed in the main body cover 111b. The through hole 1113 communicates with the through hole 1111 and holds a cover glass. The through hole 1114 communicates with the through hole 1112.

The bending portion 112 has a bending cover 112a having a flexible tubular shape. In the bending cover 112a, a plurality of bending pieces 116 having a substantially tubular shape and a tube 117 in which its one end communicates with the through hole 1112 and the other end forms the treatment tool channel 101a are provided. The tube 117 connects to the through hole 1112 through a relay member 117a. A treatment tool insertion hole 1170 is formed by a hollow space formed by the tube 117 and the through holes 1112 and 1114.

The main body cover 111b and the bending cover 112a are connected by an adhesive portion 118 that fills a step generated between both covers and connects both covers in a watertight manner.

The end Cover 200 is formed by using a light transmissive material and has a bottomed tubular shape having an opening at both ends. The end cover 200 includes a side portion 201, a bottom portion 202, and a rod portion 203. Examples of the light transmissive material include a glass and a resin that satisfies optical performance. At least the rod portion 203 may have optical transparency.

The side portion 201 has a substantially tubular shape. The bottom portion 202 is provided inside the side portion 201 and forms a bottom of the side portion 201. The bottom portion 202 has a flat plate shape perpendicular to a central axis of the side portion 201 (a central axis of the tube). The rod portion 203 extends from one main surface of the bottom portion 202 to a plane P1 that passes through a distal end of the side portion 201. It is assumed that the plane P1 is a plane perpendicular to the central axis of the side portion 201.

The end cover 200 includes a housing portion 201a (an endoscope holding portion) and an opening portion 201b. The housing portion 201a is provided on one end side of the end cover 200 on which the rod portion 203 is not formed, has a substantially concave shape formed by an inner circumferential surface of the side portion 201 and a main surface of the bottom portion 202, and can house the distal end portion 111. The opening portion 201b is provided on the other end side of the end cover 200 on which the rod portion 203 is formed, includes the through hole 1113 at least in a plan view when the end cover 200 is attached to the distal end portion 111, and forms a hollow space formed by an inner circumferential surface of the side portion 201, a main surface of the bottom portion 202, and an outer circumference of the rod portion 203. Here, “in plan view” is a case in which the end cover 200 is seen in a plane perpendicular to the central axis of the side portion 201 (tube), that is, a case in which the end cover 200 is seen in the central axis direction.

In the bottom portion 202, a through hole 202a that communicates with the through hole 1113 when the end cover 200 is attached to the distal end portion 111 is formed. On a main surface of the bottom portion 202 facing the housing portion 201a, a contact surface 202b (a probe position defining portion) is provided that is configured to come into contact with a distal end surface of the measurement probe 5 (the distal end portion 53) exposed from the distal and portion 111 through the treatment tool channel 101a.

The rod portion 203 includes at least the through hole 1114 in the bottom portion 202 in a plan view when the end cover 200 is attached to the distal end portion 111. Here, a distance between the plane P1 that passes through a distal end surface of the rod portion 203 (the side portion 201) and a plane P2 that passes through a surface of the bottom portion 202 facing the housing portion 201a (the contact surface 202b) is designed to be a distance d1 in order to maintain a constant distance between the measurement target and the distal and portion 53. The plane P2 is a plane in parallel with the plane P1.

The end cover 200 having the configuration as described above is attached to the distal end portion 111 such that the bottom portion 202 comes into contact with the main body cover 111b. In this case, the through hole 1113 and the through hole 202a communicate with each other. The through hole 1114 is located within a region in which the rod portion 203 is formed in the bottom portion 202 in plan view. A means (for example, a concave portion and a convex portion) to restrict a position where the end cover 200 is attached to the distal end portion 111 may be provided and a relative position of the end cover 200 with respect to the distal end portion 111 may be determined when the end cover 200 is attached to the distal end portion 111. Alternatively, a user may rotate the end cover 200 with respect to the distal and portion 111 and adjust the position of the through hole 1114. A probe assembly is configured by the measurement probe 5 and the end cover 200.

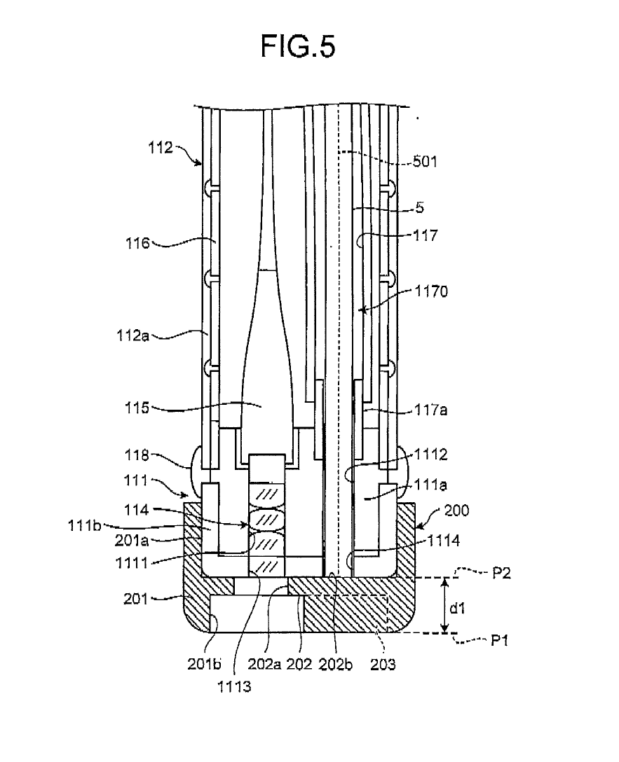

FIG. 5 is a diagram schematically illustrating a configuration of the distal and portion and the end cover of the endoscope according to the first embodiment when the measurement probe is inserted. When the measurement probe 5 is inserted from the treatment tool channel 101a in a state in which the end cover 200 is attached to the distal end portion 111, the distal and surface of the distal end portion 53 is exposed from the through hole 1114 through the treatment tool insertion hole 1170. In this case, the distal end surface of the distal end portion 53 (fiber group 501) comes into contact with the contact surface 202b of the bottom portion 202. Hence, when a measurement is performed with the measurement probe 5 by causing the end cover 200 (the distal end surface of the rod portion 203) to be in contact with the measurement target, the distance from the distal end surface of the distal and portion 53 to the measurement target becomes the distance d1 described above.

According to the first embodiment of the present invention described above, the position of the distal end portion 53 of the measurement probe 5 inserted into the treatment tool channel 101a is defined such that the distance from the position of the distal end portion 53 to the measurement target is a predetermined distance d1 by the end cover 200 attached to the distal end portion 111, so that it is possible to stabilize the relative positional relationship between the endoscope and the measurement probe.

Further, according to the first embodiment, the distal end surface of the end cover 200 (the opening surface of the opening portion 201b and the distal end surface of the rod portion 203) is in contact with the measurement target, so that it is possible to increase the contact area (contact region) in contact with the measurement target as compared with a case in which a measurement is performed by causing the distal end surface of the measurement probe 5 to be in contact with the measurement target. Therefore, it is possible to maintain a stable contact state with the measurement target.

Further, according to the first embodiment, it is possible to maintain a relative positional relationship between the imaging element 115 of the endoscope 101 and the measurement probe 5 not only in the longitudinal direction of the insertion portion 110, but also in a direction perpendicular to the longitudinal direction by aligning planes in the direction perpendicular to the longitudinal direction by the positions of distal end opening surfaces of the through holes 1113 and 1114, so that it is possible to maintain more stable measurement processing.

### Second Embodiment

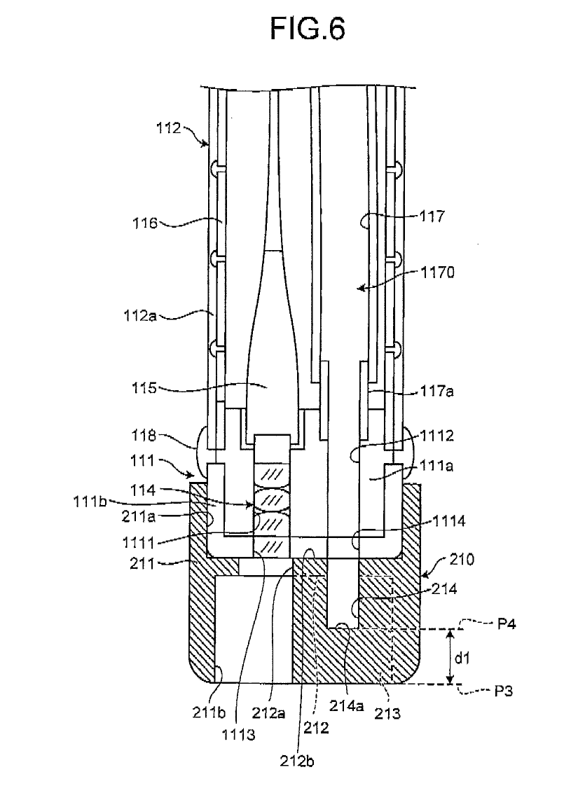

FIG. 6 is a diagram schematically illustrating a configuration of an insertion portion and an end cover of an endoscope according to a second embodiment of the present invention. The same reference signs are used to designate the same elements as those in the above-described embodiment. In the aforementioned first embodiment, the distance d1 is maintained by causing the distal end portion 111 to come into contact with the bottom portion 202 and causing the distal and portion 53 to come into contact with the bottom portion 202. However, in the second embodiment, a hole portion 214 is formed from a bottom portion 212 to a rod portion 213 and the measurement probe 5 (the distal end portion 53) is inserted into the hole portion and held.

The end cover 210 according to the second embodiment is formed by using a light transmissive material and has a bottomed tubular shape having an opening at both ends. The end cover 210 includes a side portion 211, a bottom portion 212, and a rod portion 213. In the same manner as the end cover 200 described above, the end cover 210 is formed by using a light transmissive material.

The side portion 211 has a substantially tubular shape.

The bottom portion 212 is provided inside the side portion 211 and forms a bottom of the side portion 211. The bottom portion 212 has a flat plate shape perpendicular to a central axis of the side portion 211 (a central axis of the tube). The rod portion 213 extends from one main surface of the bottom portion 212 to a plane P3 that passes through a distal end of the side portion 211.

The end cover 210 includes a housing portion 211a (an endoscope holding portion), an opening portion 211b, and the hole portion 214 (a probe position defining portion). The housing portion 211a is provided on one end side of the end cover 210 on which the rod portion 213 is not formed, has a substantially concave shape formed by an inner circumferential surface of the side portion 211 and a main surface of the bottom portion 212, and is configured to house the distal end portion 111. The opening portion 211b is provided on the other end side of the end cover 210 on which the rod portion 213 is formed, includes the through hole 1113 at least in a plan view when the end cover 210 is attached to the distal end portion 111, and forms a hollow space formed by an inner circumferential surface of the side portion 211, a main surface of the bottom portion 212, and an outer circumference of the rod portion 213.

The hole portion 214 extends from a main surface of the bottom portion 212 facing the housing portion 211a to substantially the center of the rod portion 213 in a central axis direction of the side portion 211 and forms a hollow space with a diameter in which the measurement probe 5 can be inserted. The hole portion 214 houses the measurement probe 5 (the distal end portion 53) protruded from the distal end portion 111 through the treatment tool channel 101a and includes a bottom surface portion 214a which is a contact surface that comes into contact with the distal end surface of the measurement probe 5. The bottom surface portion 214a forms a bottom surface of the hole portion 214. When the end cover 210 is attached to the distal end portion 111, the hole portion 214 communicates with the through hole 1114.

In the bottom portion 212, a through hole 212a is formed to communicate with the through hole 1113 when the end cover 210 is attached to the distal end portion 111. On the bottom portion 212 facing the housing portion 211a, a contact surface 212b is provided that is configured to come into contact with a distal end surface of the insertion portion 110.

The rod portion 213 includes at least the through hole 1114 in the bottom portion 212 in a plan view when the end cover 210 is attached to the distal end portion 111. Here, a distance between the plane P3 that passes through a distal end surface of the rod portion 213 (the side portion 211) and a plane P4 that passes through the bottom surface portion 214a is designed to be a distance d1 in order to maintain a constant distance between the measurement target and the distal and portion 53.

The end cover 210 having the configuration as described above is attached to the distal and portion 111 such that the bottom portion 212 facing the housing portion 211a comes into contact with the main body cover 111b. In this case, the through hole 1113 and the through hole 212a communicate with each other and the through hole 1114 and the hole portion 214 communicate with each other.

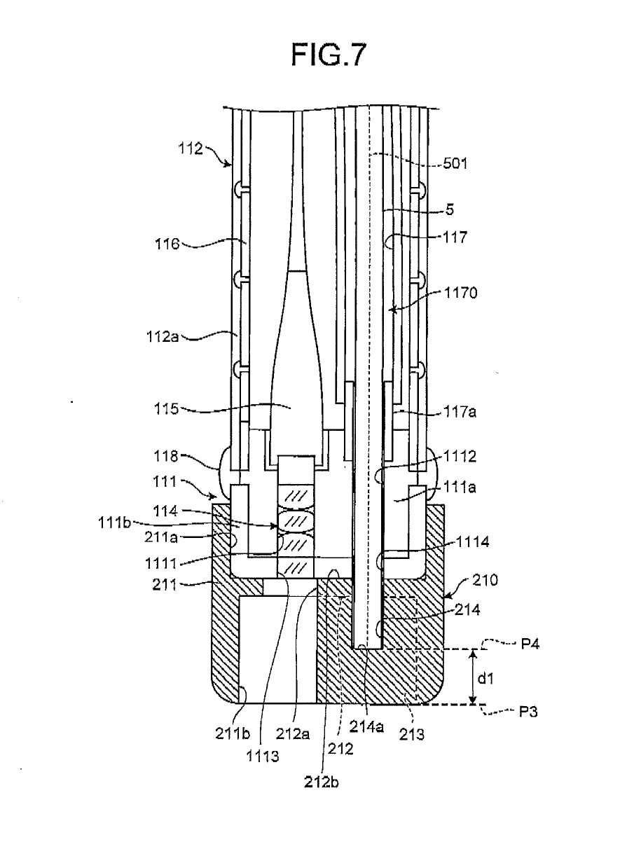

FIG. 7 is a diagram schematically illustrating a configuration of the distal end portion and the end cover of the endoscope according to the second embodiment when the measurement probe is inserted. When the measurement probe 5 is inserted from the treatment tool channel 101a in a state in which the end cover 210 is attached to the distal end portion 111, the distal end surface of the distal end portion 53 is exposed from the through hole 1114 through the treatment tool insertion hole 1170. In this case, the distal end surface of the distal end portion 53 is inserted into the hole portion 214 and comes into contact with the bottom surface portion 214a. Hence, when a measurement is performed with the measurement probe 5 by causing the end cover 210 (the distal end surface of the rod portion 213) to be in contact with the measurement target, the distance from the distal end surface of the distal and portion 53 (the fiber group 501) to the measurement target becomes the distance d1 described above.

According to the second embodiment of the present invention described above, the distal end portion 53 of the measurement probe 5 inserted into the treatment tool channel 101a is held by the end cover 210 (the hole portion 214) attached to the distal end portion 111 and the position of the distal end portion 53 is defined such that the distance from the position of the distal end portion 53 to the measurement target is a predetermined distance d1, so that it is possible to stabilize the relative positional relationship between the endoscope and the measurement probe.

Further, according to the second embodiment, the distal end surface of the end cover 210 (the opening surface of the opening portion 211b and the distal end surface of the rod portion 213) is in contact with the measurement target, so that it is possible to increase the contact area in contact with the measurement target as compared with a case in which a measurement is performed by causing the distal end surface of the measurement probe 5 to be in contact with the measurement target. Therefore, it is possible to maintain a stable contact state with the measurement target.

### First Modified Example of Second Embodiment

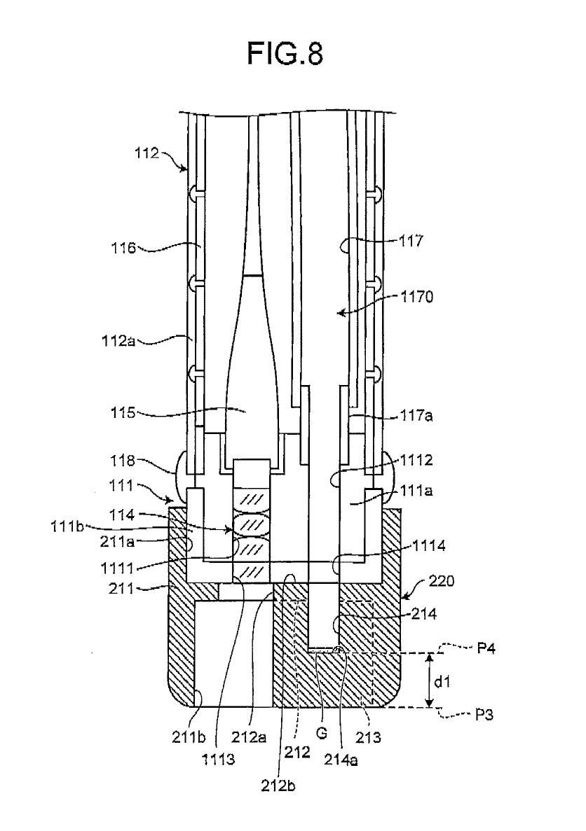

FIG. 8 is a diagram schematically illustrating a configuration of an insertion portion of an endoscope, a distal end portion of a measurement probe, and an end cover according to a first modified example of the second embodiment of the present invention. In the second embodiment described above, the distal end surface of the distal end portion 53 is configured to directly come into contact with the bottom surface portion 214a of the hole portion 214. However, in the first modified example, a gel G is arranged on the bottom surface portion 214a.

The end cover 220 illustrated in FIG. 8 is provided with the gel G on the bottom surface portion 214a of the hole portion 214 in addition to the configuration of the end cover 210 described above. The gel G is formed of a gel-like substance having optical transparency. Besides the gel-like substance, a resin or the like having optical transparency and elasticity may be used.

In the same manner as in the second embodiment described above, the and cover 220 having the configuration as described above is attached to the distal end portion 111 such that the bottom portion 212 comes into contact with the main body cover 111b. When the measurement probe 5 is inserted from the treatment tool channel 101a in a state in which the end cover 220 is attached to the distal end portion 111, the distal end surface of the distal and portion 53 is protruded from the through hole 1114 through the treatment tool insertion hole 1170 and inserted into the hole portion 214. At this time, the distal end surface of the distal end portion 53 comes into contact with the bottom surface portion 214a of the hole portion 214 through the gel G. Hence, when a measurement is performed with the measurement probe 5 by causing the end cover 220 (the distal end surface of the rod portion 213) to be in contact with the measurement target, the distance from the distal end surface of the distal end portion 53 to the measurement target can be substantially the same as the distance d1 described above and the distal end portion 53 and the end cover 220 can be in close contact with each other in a contact portion thereof. Hence, it is possible to suppress decrease in connection efficiency of the contact portion between the distal end portion 53 and the end cover 220.

### Second Modified Example of Second Embodiment

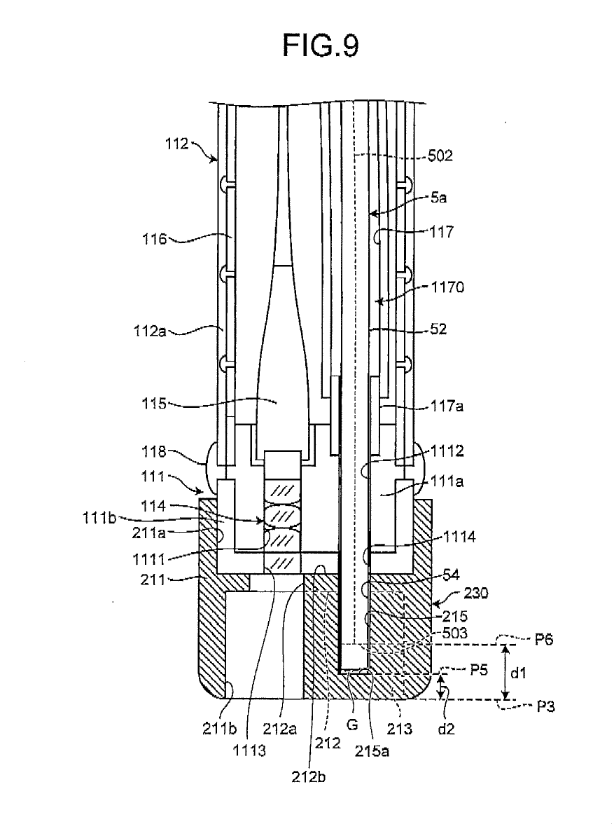

FIG. 9 is a diagram schematically illustrating a configuration of an insertion portion of an endoscope, a distal and portion of a measurement probe, and an end cover according to a second modified example of the second embodiment of the present invention. In the second embodiment described above, the distal end surface of the distal end portion 53 from which the fibers are exposed is configured to come into contact with the bottom surface portion 214a of the hole portion 214. However, in the second modified example, a measurement probe 5a includes a distal end portion 54 having a glass rod 503.

The measurement probe 5a includes a proximal end portion 51 (not illustrated in FIG. 9) that is attachably and detachably connected to the connector unit 22 of the optical measurement device 2, a flexible portion 52 having flexibility, and a distal end portion 54 in which the glass rod 503 is exposed from its distal end surface and which emits the illumination light supplied from the light source unit 21 through the connector unit 22 and receives the return light of the illumination light from the measurement target. In the distal end portion 54, a fiber group 502 including an illumination fiber and light receiving fibers is fixed in a state of being in contact with the glass rod 503.

In the end Cover 230 illustrated in FIG. 9, a hole portion 215 (a probe position defining portion) is formed instead of the hole portion 214 of the end cover 210 described above. Further, a gel G is provided on a bottom portion 215a of the hole portion 215. In the hole portion 215, a distance d2 from a plane P5 that passes through the bottom portion 215a to the plane P3 is smaller than the distance d1 from the plane P3 to the plane P4 described above by a length of the glass rod 503. In the second modified example, it is designed so that a distance from the plane P3 to a plane P6 that passes through a contact surface of the glass rod 503 with which the illumination fiber and the light receiving fibers come into contact is d1 described above.

In this way, even when the glass rod 503 is provided at the distal end portion of the measurement probe Sa, it is possible to stabilize the relative positional relationship between the endoscope and the measurement probe and it is also possible to set the distance to the measurement target to a predetermined distance.

### Third Embodiment

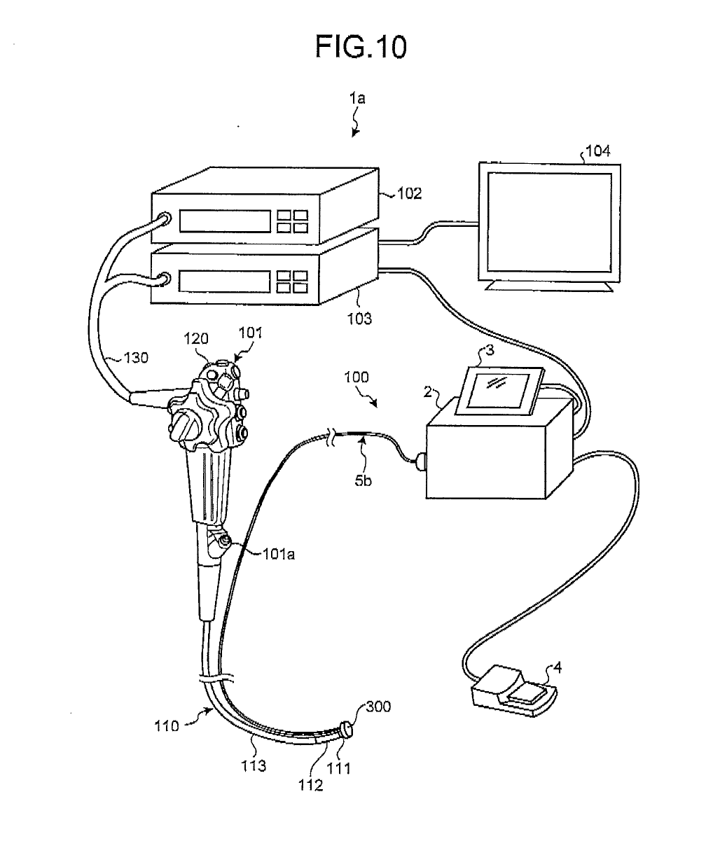

FIG. 10 is a schematic diagram illustrating a schematic configuration of an endoscope system according to a third embodiment of the present invention. The same reference signs are used to designate the same elements as those in FIG. 1 and the like. In the aforementioned first embodiment, the measurement probe 5 is inserted into the treatment tool channel 101a. However, in an endoscope system 1a according to the third embodiment, a measurement probe 5b is held by an end cover 300 independently from the endoscope 101. In other words, a probe assembly including the measurement probe 5b and the and cover 300 holds the endoscope 101.

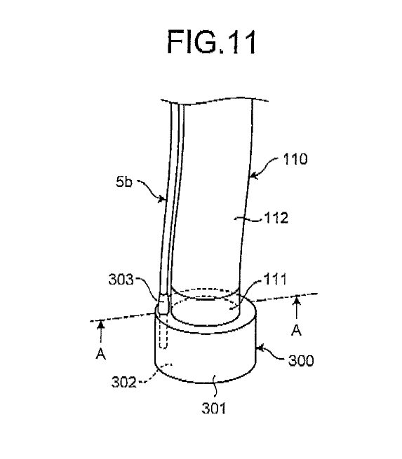

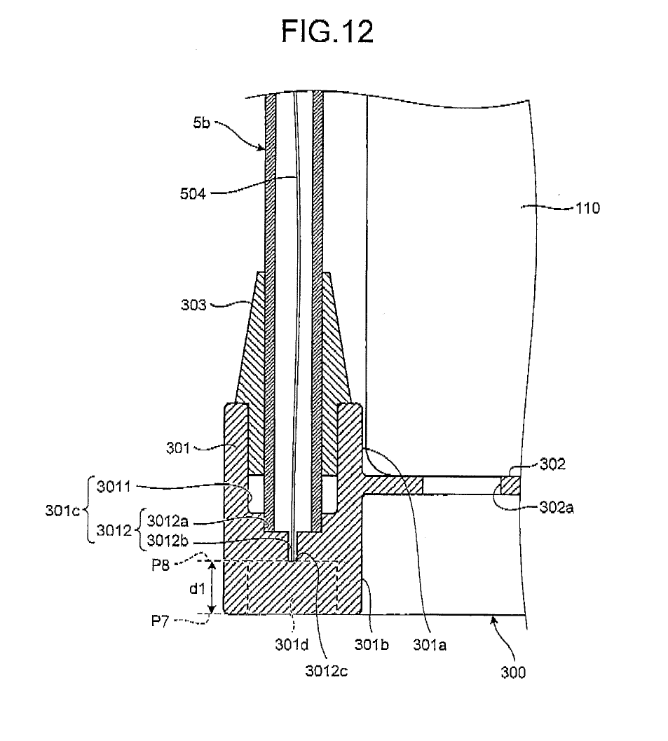

FIG. 11 is a perspective view schematically illustrating a configuration of an insertion portion of the endoscope, a distal end portion of the measurement probe, and the end cover according to the third embodiment. FIG. 12 is a partial cross-sectional view taken along line A-A in FIG. 11 and is a diagram illustrating a part of a configuration of the insertion portion of the endoscope, the distal end portion of the measurement probe, and the end cover. The end cover 300 is formed by using a light transmissive material and has a bottomed tubular shape having an opening at both ends. The end cover 300 includes a side portion 301, a bottom portion 302, and a fixation member 303.

The side portion 301 has a substantially tubular shape. The bottom portion 302 is provided inside the side portion 301 and forms a bottom of the side portion 301. The bottom portion 302 has a flat plate shape perpendicular to a central axis of the side portion 301 (a central axis of the tube).

The end cover 300 includes a housing portion 301a (an endoscope holding portion), an opening portion 301b, a probe holding portion 301c (a probe position defining portion), and a rod portion 301d. The housing portion 301a is provided on one end side of the end cover 300, has a substantially concave shape formed by an inner circumferential surface of the side portion 301 and a main surface of the bottom portion 302, and can house the distal end portion 11. The opening portion 301b is provided on the other end side of the end cover 300 and has a substantially concave shape formed by an inner circumferential surface of the side portion 301 and a main surface of the bottom portion 302.

In the bottom portion 302, a through hole 302a that communicates with the through hole 1113 when the end cover 300 is attached to the distal end portion 111 and a through hole (not illustrated) which communicates with the through hole 1114 and into which a treatment tool that is exposed through the treatment tool channel 101a can be inserted are formed.

The probe holding portion 301c includes: a first hole portion 3011 that is a hole forming a cylindrical hollow space from a surface of the end cover 300 facing the housing portion 301a; and a second hole portion 3012 that is a bottom of the probe holding portion 301c. The second hole portion 3012 has a diameter similar to that of a distal end surface of the measurement probe 5b and includes a first holding portion 3012a that holds the measurement probe 5b and a second holding portion 3012b that extends with a diameter smaller than that of the first holding portion 3012a and holds a fiber group 504 including an illumination fiber and light receiving fibers. The second holding portion 3012b has a bottom surface portion 3012c which is a contact surface that comes in contact with a fiber distal end surface of the fiber group 504. The first hole portion 3011 has a diameter greater than that of the first holding portion 3012a of the second hole portion 3012.

The rod portion 301d extends from the bottom surface portion 3012c to a plane P7 passing through a distal end of the side portion 301. A distance between the plane P7 and a plane P8 passing through the bottom surface portion 3012c is designed to be a distance d1 in order to maintain a constant distance between the measurement target and the distal end portion 53.

The fixation member 303 has a substantially cylindrical shape. The diameter of the inner circumference of the fixation member 303 is similar to that of the distal and portion of the measurement probe 5b. The fixation member 303 is formed by using an elastically deformable material.

One end of the fiber group 504 is connected to the light source unit 21 and the imaging unit 23 through the connector unit 22 and the other end is fixed to the second hole portion 3012 in a state of being in contact with the bottom surface portion 3012c. The distal end of the measurement probe 5b is held by the fixation member 303. The arrangement of the measurement probe 5b is fixed when the fixation member 303 is pressed into the first housing portion 301a. The measurement probe 5b is held through the fixation member 303, so that it is possible to prevent the fibers from being folded at the distal end of the measurement probe 5b.

According to the third embodiment of the present invention described above, the end cover 300 to which the insertion portion 110 (the distal end portion 111) can be attached holds a distal end portion of the measurement probe 5b, and the position of the fiber group 504 exposed from the distal end portion is defined such that the distance from the position of the fiber group 504 to the measurement target is a predetermined distance d1, so that it is possible to stabilize the relative positional relationship between the endoscope and the measurement probe.

According to some embodiments, it is possible to stabilize the relative positional relationship between the endoscope and the measurement probe.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A probe assembly comprising:
a measurement probe having a plurality of optical fibers configured to propagate light, emit the light to a measurement target, and receive scattered light returned from the measurement target; and
an end cover having: an endoscope holding portion configured to hold an insertion portion of an endoscope, the insertion portion being configured to be inserted into a body cavity of a subject; and a probe position defining portion configured to define a position of a distal end surface of the plurality of optical fibers of the measurement probe.

2. The probe assembly according to claim 1, wherein the probe position defining portion is a contact surface of the endoscope holding portion, which is configured to come into contact with a distal end of the insertion portion and to come into contact with a distal end surface of the measurement probe.

3. The probe assembly according to claim 2, wherein the contact surface is configured to come into contact with the distal end surface of the plurality of optical fibers.

4. The probe assembly according to claim 2, further comprising a light transmissive member that is provided at a distal end of the measurement probe and has one end configured to come into contact with the distal and surface of the plurality of optical fibers, wherein
the contact surface is configured to come into contact with the other end of the light transmissive member.

5. The probe assembly according to claim 1, wherein the probe position defining portion is a hole which is configured to house a distal end of the measurement probe and which has a bottom surface portion configured to come into contact with a distal end surface of the measurement probe.

6. The probe assembly according to claim 5, wherein the hole is formed in a part of a contact surface of the endoscope holding portion, which is configured to come into contact with the insertion portion.

7. The probe assembly according to claim 5, wherein the hole is formed separately from the endoscope holding portion.

8. An end cover comprising:
an endoscope holding portion configured to hold an insertion portion of an endoscope, the insertion portion being configured to be inserted into a body cavity of a subject; and
a probe position defining portion configured to define a position of a distal end surface of a plurality of optical fibers of a measurement probe, the plurality of optical fibers being configured to propagate light, emit the light to a measurement target, and receive scattered light returned from the measurement target.
